(19) United States
(12) Patent Application Publication
(10) Pub. No.: US 2010/0305604 A1
(43) Pub. Date: Dec. 2, 2010

(54) Device for Filtering Blood

(76) Inventor: Gunnar Pah, Hoervik (NO)

Correspondence Address: HOUSTON ELISEEVA, 420 BEDFORD ST, SUITE 155, LEXINGTON, MA, 02420, US

(21) Appl. No.: 12/744,883

(22) PCT Filed: Nov. 27, 2008
(86) PCT No.: PCT/EP08/66297

§ 371 Date: Jul. 26, 2010

(30) Foreign Application Priority Data
Nov. 27, 2007 (DE) 10 2007 056 946.9

(51) Int. Cl. A61F 2/01

(52) U.S. Cl. 606/200

(57) ABSTRACT

The device for the filtering of blood comprises a filter catheter with a filter at its distal end for filtering the blood branching downstream behind the heart valve into the arteries to supply the brain and the arms, wherein the filter may be pushed out of a catheter tube in the distal direction. The filter is tubular or designed to taper from the distal to the proximal end. The filter is designed to fit tightly, at least at its distal end section, against the ascending aorta in the area upstream of the arteries which supply the brain and the arms.

The present invention relates to a device for the filtering of blood. The filter is provided for filtering the blood which branches off in the aortic arch to supply the brain and the arms.

In the as yet unpublished German patent application DE 10 2006 024 179, a device for the filtering of blood in the removal of a heart valve stenosis is described. This comprises a filter catheter which has a filter at its distal end for filtering the blood which is located downstream behind a heart valve. The filter may be pushed distally out of and into the filter catheter. The filter is designed to expand radially in such a way that in the extended state it extends radially out from the filter catheter and makes contact with a blood vessel wall. The filter catheter has a catheter lumen which is so designed that a balloon catheter may be guided through the catheter lumen. With the balloon catheter, a valvuloplasty may be performed, with the downstream filter filtering plaque, debris and embolism-triggering material from the blood.

Known from WO 99/23976 is a catheter with a folding filter element. The filter element is mounted on a filter support and is designed for feeding through the patient's system of blood vessels. The filter element may be transferred from a folded storage position in the filter support, for movement through the blood vessel system, into an unfolded position in which a blood vessel is closed off, so that blood flowing through the blood vessel is guided through the filter element. The filter element comprises a folding filter body with an inlet end and an outlet end, wherein the inlet end of the filter body has one or more openings which allow blood containing embolism-triggering material, plaque and debris to enter the filter body. The outlet end of the filter body has a multiplicity of outlet openings which are so designed that blood is able to pass through, but undesired embolic material is retained within the filter body. In addition, means are provided for closing the inlet opening and the outlet opening of the filter body. These openings are closed before the filter is closed prior to withdrawal. This published document proposes a multiplicity of filter elements for this purpose.

Known from WO 00/67668 is another filter element with a folding filter body. The filter body may be transferred from a folded position in which it may be moved through a cardiovascular system into an unfolded position, in order to extend into a blood vessel. In the unfolded position, blood flowing through the blood vessel is guided through the filter element and filtered. A distal end section of the filter body has one or more inlet openings, which are so dimensioned that blood, embolism-causing material, and plaque and debris are able to enter the filter body, and a proximal outlet section which has many small outlet openings which are so dimensioned that blood may pass through them, but embolic material, plaque and debris are retained. The filter body is at least partly a laminate construction, having a membrane provided with a coating which is bio-compatible.

U.S. Pat. No. 6,676,683 B1 discloses a filter catheter which has at its end section an extendable and expandable filter. The catheter itself is a wire catheter along which other catheters which comprise this wire catheter may be guided, for suitable positioning in the blood vessels.

EP 980 278 B1 discloses a similar catheter with a guide wire, on the end of which an expandable filter element is provided.

The disadvantage of the filters known from the prior art is that they affect the flow of blood and create a considerable backpressure in the blood vessel.

An artificial heart valve prosthesis is known from e.g. EP 1 335 683 B1. Other implantable heart valve prostheses, together with catheters for the implantation of such heart valve prostheses, are disclosed by EP 592 410 B1, US 2004/0210304 A1, U.S. Pat. No. 7,018,406 B2, WO 2006/127765 A1, US 2003/0036795 A1, U.S. Pat. No. 5,411,52, U.S. Pat. No. 6,168,614 B1, U.S. Pat. No. 6,582,462 B1 and WO 91/17720.

Known from DE 1 988 277 T1 is a catheter with a folding filter body. The filter body may be pushed out of the distal end of the catheter. The filter body has a proximal inlet which expands from the catheter to a filter in the shape of a funnel. The inlet has several inlet openings, through which blood and embolic material can penetrate into the filter element. The filter body includes a tubular section with a distal outlet. The outlet has several outlet openings which are so designed that blood flows though, while undesired embolic material is retained within the body of the filter element. The proximal inlets are larger than the distal outlets.

Known from WO 03/017823 A2 is a filter catheter which is inserted in an artery in the direction of flow. The filter similarly has a tubular section and a funnel-shaped tapering section. The tapering section is formed at the distal end of the filter, and the funnel-shaped section at the proximal end of the filter. The proximal section has openings through which undesired material may penetrate into the filter.

WO 2006/116636 discloses a self-expanding stent with filter body. The filter body may be formed in the distal end of the stent, and has at its distal end pores which allow blood to pass through, but hold back embolism-causing material or larger particles.

An implantable medical device is described in WO 2007/067451. The device has a tubular section in the form of a stent, and a funnel-shaped section in the form of a filter. Blood is able to flow through the device in both axial directions, and may be introduced into the body with or against the direction of blood flow.

Described in WO 2007/133887 A1 is a “thimble-like” filter catheter which may be inserted into the artery in the direction of the blood flow. The filter may have filter openings of equal size throughout its length.

Described in US 2007/0203559 A1 is a stent with a proximal end and a distal end. The stent has a kind of bell shape, with an expanded distal end and a funnel-shaped proximal end.

The invention is based on the problem of creating a device and a method for the filtering of blood, with which embolism-causing material, plaque and debris are reliably retained, and the influence of the filter on blood flow and the resulting backpressure is kept to the minimum.

The problem is solved by a device with the features of claim 1 or claim 14 and a method with the features of claim 16. Advantageous developments are set out in the relevant dependent claims.

The device according to the invention for the filtering of blood comprises a filter catheter with a filter at its distal end for filtering the blood branching downstream behind the heart valve into the arteries to supply the brain and the arms. This filter may be pushed out of a catheter tube in the distal direction and the filter is designed to expand from the proximal to the distal in the extended state. The filter has a distal filter section and a proximal section, and the distal filter section has filter openings, and the proximal section has one or more openings which are larger than the filter openings of the distal filter section.

The provision of larger openings in the proximal section keeps backpressure to a minimum and reduces stress on the sick heart. In addition, the force exerted by the blood pressure on the filter is kept low, thereby avoiding unintentional shifting of the distal end of the filter in the direction of flow.

It has been shown that, despite almost complete opening of the proximal section, sufficient blood can be filtered in the aortic arch and branched off to the brain. Moreover, the larger openings in the proximal section ensure an adequate supply to the remainder of the body, even when the functioning ability of the heart is restricted.

The section in the aortic arch in which the arteries to supply the brain and the arms branch off is described below as the aortic arch section. The arteries to supply the brain and the arms are the truncus brachiocephalicus, which divides into the right subclavian artery (arteria subclavia dextra) and the right common carotid artery (arteria carotis communis dextra), the left common carotid artery (arteria carotis communis sinistra) and the left subclavian artery (arteria subclavia sinistra). According to one embodiment, the filter has a tubular distal filter section and a proximal section, which becomes narrower from the distal filter section to the catheter tube. The filter is longer than the aortic arch section and is so designed that, in the area upstream of the aortic arch, it fits tightly against the ascending aorta at its distal end section. Filter openings are formed in the distal filter section. The proximal section has one or more openings, which are larger than the filter openings of the distal filter section.

Due to the fact that the filter openings of the filter in the area of the bifurcations to the arteries which supply the brain and the arms are made relatively small, very fine particles can be filtered from the blood, thus preventing these particles from gaining access to the brain via the arteries supplying the brain and the arms, and thereby preventing a stroke.

No particles or only larger particles are filtered by the proximal filter section. Smaller particles do not represent a high relevant health risk in blood flowing in the legs. The backpressure in conventional filter catheters, on the other hand, stresses the heart considerably, which can be extremely problematic in operations of this kind.

According to a further embodiment of the present invention, the device comprises a filter catheter which, to filter the blood branching off downstream behind the heart valve in the aortic arch into the arteries supplying the brain and the arms, has at its distal end an elongated filter tapering from the distal to the proximal. At its proximal end, the filter merges into a catheter tube and may be pushed distally out of the catheter. The filter is longer than the aortic arch section at which the arteries to supply the brain and the arms branch off, and is so designed that it fits tightly against the ascending aorta, at least with its distal end section, in the area upstream of this section.

Due to the elongated design of the filter and the nature of its arrangement in the area of the aortic arch, a large filter surface is provided. Because of the large filter surface of the tapering filter, the backpressure of blood is kept to a minimum.

The filter is so designed that the filter openings of the filter increase in size from the distal to the proximal end, for example over three sections. By this means it is ensured that the blood flowing into the arteries to supply the brain and the arms is finely filtered. Because of the larger filter openings at the proximal end, the flow of blood is virtually unimpeded.

According to another aspect of the invention, the two embodiments described above may also be provided with filter openings which have a roughly constant size over the entire length of the filter, with a diameter in the range of approximately 150 μm to 250 μm and preferably 150 μm to 200 μm. This opening size is not only small enough to filter particles from blood flowing out of the brain, but is also still large enough to keep backpressure to a minimum. This applies in particular to the variant of the filter which tapers gradually from distal to proximal, since here a very large filter surface is provided. An opening size which increases towards the proximal end of the filter is however preferred.

In the method according to the invention, a filter catheter is used. The filter catheter is fitted a short distance downstream of the heart valve in the aortic arch in the area where the arteries supplying the brain and the arms branch off.

The filter catheter may be inserted in the body by all known means of access, and guided through the ascending aorta to the aortic arch. For example the filter catheter is inserted into the body through the femoral artery or the radial artery or the subclavian artery.

The filter catheter is so designed that it may be inserted into the body against the direction of the blood flow.

In the extended state the filter is so designed that embolism-causing material, plaque and debris collecting in the filter are taken into the catheter lumen when the filter is withdrawn into the catheter tube, and do not remain in the aorta. In comparison with conventional filters, the filter is much longer, so that it defines a large volume in which particles to be filtered may accumulate. When the filter is withdrawn into the catheter tube, these particles accompany the filter into the catheter tube and are safely removed from the body.

To extend the filter, various mechanisms are possible according to the invention. The most simple mechanism according to the invention provides for the filter to be connected to a filter wire or similar device, by which it is pushed into or pulled out of the catheter tube. The provision of a filter element, which acts accordingly, is also possible.

The filter and/or the filter wire may be provided with an anti-thrombogenic coating, to prevent the adhesion of blood or clotting of the blood.

In a further advantageous embodiment the catheter tube is made of two tube-like coaxial bodies, with the inner body connected permanently to the filter. By moving the inner body within the outer body, the filter may be withdrawn from or inserted into the outer body.

Instead of an outer and an inner body, the catheter tube may also be made of a single-piece, hollow-cylindrical or tubular body. Formed in the wall of this body are hollow passages running from the proximal to the distal end, each containing a filter wire. Provided at the distal end of this body is a recess to accommodate the filter. At its proximal end the filter is fastened to a ring. The ring in turn is fixed to the ends of the filter wires. By means of the filter wires, the filter may therefore be pushed out of the catheter tube and drawn back into the catheter tube. The filter wires are all actuated simultaneously, to avoid tilting of the filter in the catheter tube. Preferably around three to five filter wires are provided, each spaced apart from one another at the same angle in the catheter tube.

The diameter of the filter catheter is roughly 8 to 24 french (FR) (approx. 2.5 mm to 8 mm); french is a unit of size common in the field of catheters.

The filter may be extended to a diameter of 120 french (approx. 40 mm) up to 180 french (approx. 60 mm). The outside diameter, at least of the distal end section of the filter, is greater than that of the aorta, in order to create radial stiffness or adhesion through frictional and/or positive engagement, so that the filter is fixed in the aorta.

Preferably the filter is located in the aortic arch at the bifurcations to the arteries which supply the brain and the arms. The risk of a stroke is advantageously reduced by the filtering of the blood which branches off into the arteries supplying the brain and the arms.

The catheter lumen is so designed that a balloon catheter may be guided through it. The present invention is not however limited to the guiding through of a balloon catheter. Other catheters may also be guided through.

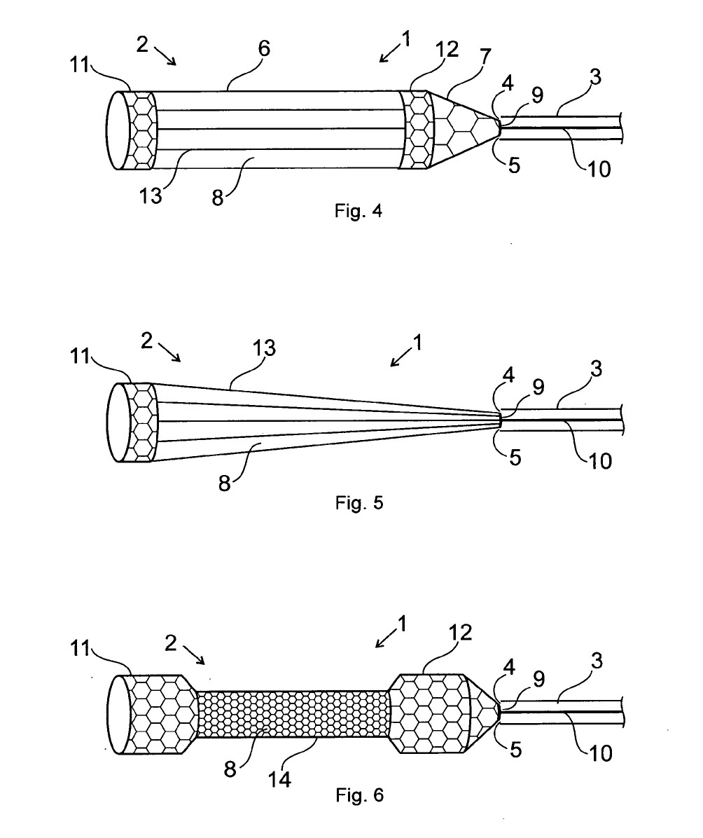

The filter catheter according to the invention may also be used in connection with a surgical perforation of the ascending aorta (see e.g. FIG. 4 of U.S. Pat. No. 6,676,683 B1), in which e.g. embolic material is intended to be removed from a blood vessel.

Reference is made in full to the contents of DE 10 2006 024 179 and PCT/EP2007/054777, and these documents are included in the present application.

The invention will be explained by way of example with the aid of the drawings, which show in schematic form in:

FIG. 1 the arrangement in the aortic arch of the filter catheter according to the invention

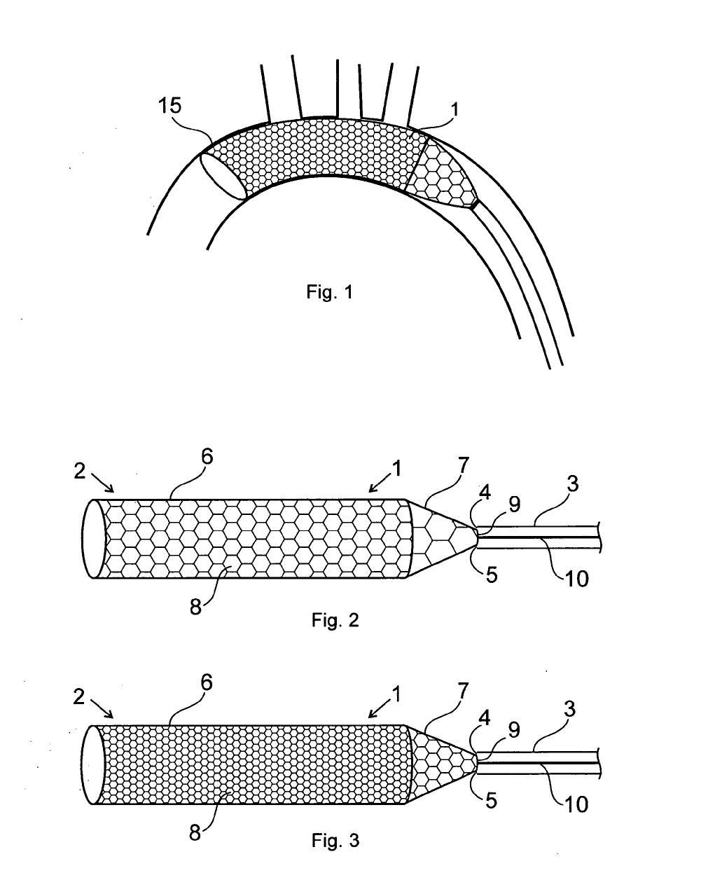

FIG. 2 a first embodiment of the filter catheter with a cut-open catheter tube

FIG. 3 a second embodiment of the filter catheter with a cut-open catheter tube

FIG. 4 a third embodiment of the filter catheter with a cut-open catheter tube

FIG. 5 a fourth embodiment of the filter catheter with a cut-open catheter tube

FIG. 6 a fifth embodiment of the filter catheter with a cut-open catheter tube

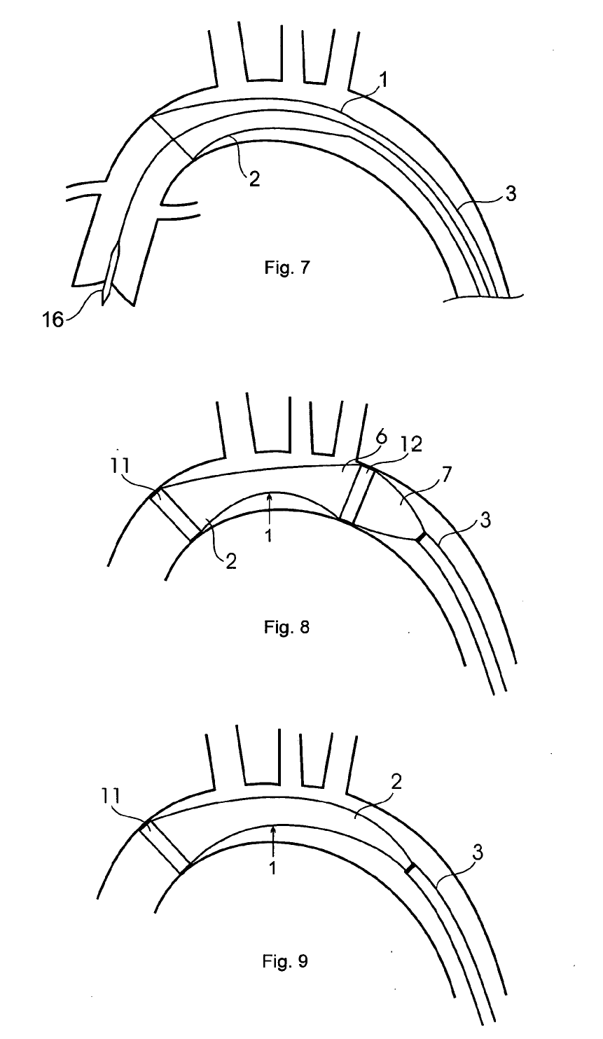

FIG. 7 a filter catheter according to the invention together with a balloon catheter in a schematic sectional view

FIG. 8 the arrangement of the filter catheter according to FIG. 4 in the aortic arch, and

FIG. 9 the arrangement of the filter catheter according to FIG. 5 in the aortic arch.

The invention is explained with the aid of several embodiments. Identical parts are provided with the same reference numbers. Unless otherwise stated, they have the same properties as the embodiments explained previously.

The device 1 according to the invention is a filter catheter (FIGS. 2-9). The filter catheter 1 has a filter 2 and a catheter tube 3, while the filter 2 is comprised of a catheter lumen 5 which may be pushed out at the distal end 4 of the catheter tube 3.

The filter 2 according to a first embodiment has a distal filter section 6, which is hollow-cylindrical or tubular in form, and a proximal section 7 which tapers from the distal filter section to the catheter tube 3 (FIG. 2). The distal filter section 6 has a length of around 3 cm to 15 cm, or a length of around 5 cm to 14 cm, or a length of around 6 cm to 14 cm, or a length of around 7 cm to 14 cm, or a length of around 8 cm to 14 cm, and preferably a length of around 10 cm to 14 cm and a diameter in the expanded state of around 3 cm to 6 cm. The proximal section 7 has a length of around 3 cm to 5 cm.

In the present embodiment, the proximal section 7 is conical. It may however also be convex or concave in cross-section, or have another tapering form.

The filter 2 is made from a lattice structure of memory material, e.g. nitinol, or another suitable memory alloy, e.g. a ferrous alloy, a copper alloy, or another memory material, e.g. plastic. The lattice structure is designed so that it supports the filter 2 as it expands during pushing out of the catheter 3. This means that the lattice struts of the lattice structure are under preload in the compressed state in the catheter lumen 5 of the catheter tube 3. On pushing out of the catheter lumen 5, the lattice structure relaxes, assuming its preset shape and in this way supporting the expansion of the filter.

Provision may be made to cool the filter before inserting it in the body, so that it heats up because of the body temperature on pushing out of the catheter tube 3. In this way the memory material of the lattice structure assumes the expanded form and thus supports the unfolding action.

The lattice structure or the struts of the filter may be provided with an anti-thrombogenic coating, to prevent the adhesion of blood. Such coatings are produced by e.g. the company SurModics/USA and the company Carmeda/Sweden. Omega-3 fatty acids and heparin are typical base substances of the anti-thrombogenic coating.

Provided as filter element 8 is a plastic film, e.g. of polyurethane (PU) or another suitable plastic. The film is arranged to fit up against the inside of the lattice structure to which it is bonded. The pores of the filter have a smaller diameter in the distal filter section 6 than in the proximal section 7. In the area of the distal filter section 6, the film has pores with a diameter of around 100 μm to 300 μm and preferably between 120 μm and 200 μm. In the proximal section 7, pores with a diameter of around 150 μm to 400 μm and preferably between 200 μm and 300 μm are provided.

The plastic film may also be provided in the area between the meshes of the lattice structure or on the outside of the lattice structure. Other suitable materials for the filter element 8 are for example Teflon (PTFE), polypropylene (PP) and polyethylene (PE). The filter element may also encompass the lattice structure of the filter completely, in order to prevent any contact between the lattice structure and the blood. In this way, adhesion or clotting of the blood on the lattice structure of the filter is prevented.

It is also possible to provide for the proximal section 7 to have no filter element 8, but instead only approximately 4 to 10 peripherally arranged struts or a lattice structure. With this arrangement, the main blood flow is virtually unimpeded.

In a healthy person, roughly 20% of the entire blood flow is branched off in the aortic arch to supply the brain and the arms. The remaining 80% flows on through the descending aorta and supplies the rest of the body.

Tests on blood flow models have shown that with a filter 2 which has only a distal filter section 6 and is not provided with a filter element 8 in the proximal section 7, an adequate amount of blood of approximately 20% of the entire blood flow passes via the distal filter section 6 into the arteries which supply the brain and the arms. The blood flow which branches off may therefore be filtered very finely, but sufficient blood still passes into the arteries which supply the brain and the arms, and this occurs even with virtually no flow resistance at the proximal end of the filter 2.

At least in its distal end section, the distal filter section 6 of the filter 2 lies in the expanded state, in the area 15 upstream of the arteries which supply the brain and the arms, tightly against the vessel wall of the ascending aorta. It is therefore expedient to provide the lattice structure of the distal end section of the distal filter section 6 with greater stiffness in the radial direction than in the remainder of the filter section 6. Such increased stiffness in this area may be obtained for example through an enlarged diameter of the lattice structure, thicker lattice struts which are then under greater preload in the catheter lumen, a continuous convex expansion in the radial direction, a radially expanding design, or other suitable measures.

The remainder of the section of the distal filter section 6 extending in the proximal direction or its lattice structure may be so designed that high stiffness is provided in the axial direction.

The distal filter section 6 of the filter 2 may be made tubular in such a way that it lies flat against the vessel wall of the aortic arch in the area of the bifurcations to the arteries supplying the brain and the arms. The bifurcations to the arteries which supply the brain and the arms are fully covered by the filter element 8 of the filter 2. In this way, blood branching off into the coronary arteries is finely filtered through the smaller pores in the distal filter section 6 of the filter 2. By this means, even very small impurities are filtered from the blood and are not able to reach the brain.

The through flow or main flow of the blood is less intensively filtered by the wider-meshed filter element 8 or the larger openings in the proximal section 7 of the filter 2. The proximal section 7 of the filter 2 gives only a low level of flow resistance. The backpressure of blood is kept to a minimum by this means, while simultaneously optimal filtering of the blood branching off into the coronary arteries is obtained.

The proximal end of the proximal section 7 is connected via an end ring 9 to a filter wire 10, which extends from the distal to the proximal end, with the ability to move through the catheter lumen 5 of the catheter tube 3. The end ring 9 is made of a radiopaque material such as e.g. steel, nitinol or gold.

The end ring 10 or its position may be detected by means of X-rays. Using this detection of the end ring 9, the position of the filter catheter 1 and its arrangement in the aortic arch may be monitored. It is also possible to provide markers, e.g. of gold, in the distal end section of the distal filter section 6, so that the distal end of the filter section 6 may also be detected using X-rays.

By moving the filter wire 10 within the catheter tube 3 from the proximal to the distal end, the filter 2 may be pushed out of the catheter tube, so that it will unfold on account of the material and design of the lattice structure.

In a second embodiment of the present invention, the filter 2 has a form similar to that of the first embodiment, with a proximal section 7 and a distal filter section 6 (FIG. 3). The filter 2 is again made of a nitinol lattice structure, which has a fine enough mesh for the lattice structure to form the filter element 8. The size of the openings corresponds to that of the embodiment described above, while the lattice structure may similarly be provided with an anti-thrombogenic coating.

In a third embodiment of the present invention, a distal filter section 6 and a proximal section 7 are also provided (FIGS. 4, 8). At the distal and proximal ends of the distal filter section 6, a distal ring section 11 and a proximal ring section 12 are provided in each case. The ring sections 11, 12 are made of nitinol. The two annular sections 11, 12 are joined together by struts 13 extending in the axial direction.

In the present embodiment, the ring sections 11, 12 are formed by an annular lattice structure which has the necessary strength to fit tightly against the blood vessel walls.

It is also possible to provide the lattice structures of the ring sections 11, 12 with a greater diameter than the section 7, or to design them with a continuous convex expansion in the radial direction, or to make the ring sections 11, 12 each expand radially at their distal or proximal end section.

The ring sections 11, 12 are made of a memory material and provided with a filter element 8.

The distal ring section and the proximal ring section 11, 12 have a length of around 0.1 cm to 3 cm.

The struts or wire-like spacers 13 are spaced roughly evenly apart from one another. Around 4 to 10 struts 13 are provided along the periphery of the distal and proximal ring sections 11, 12. The struts 13 are made of a memory material such as e.g. nitinol, and have a length of around 8 cm to 14 cm.

Located on the insides of the struts 13 and on the inside of the lattice structure of the proximal section 7 is a plastic film as filter element 8. The plastic film has in the area of the struts 13 filter openings with a diameter of around 100 μm to 200 μm. Provided in the proximal section 7 are filter openings with a diameter of around 200 μm to 300 μm. For the reasons given above, it is provided for the filter openings in the area of the struts 13 to have a smaller diameter than the filter openings in the proximal conical section 7.

Provided at the proximal end of the proximal section 7 is a radiopaque end ring 9 which is connected to the filter wire 10.

It is also possible to provide for the distal and/or proximal ring section 11, 12 to be made of a lattice structure serving as filter element 8, in accordance with the second embodiment.

In the two embodiments just described, provision is made for the filter openings of the plastic film in the distal filter section 6 to be smaller than the filter openings in the proximal section 7.

It may also be provided that the proximal section 7 is formed of axially extending struts or a lattice structure with large openings.

In a fourth embodiment of the present invention, the filter catheter 1 has an elongated filter 2 which is funnel-shaped or tapers from the distal to the proximal end (FIGS. 5, 8).

The filter has a length of around 5 cm to 16 cm or a length of around 6 cm to 15 cm or a length of around 5 cm to 14 cm or a length of around 6 cm to 14 cm or a length of around 7 cm to 14 cm or a length of around 8 cm to 14 cm and preferably a length of around 10 cm to 14 cm, and a diameter in the expanded state in the distal end section of around 3 cm to 6 cm.

Provided at the distal end of the filter 2 is a ring section 11 of nitinol. The ring section 11 may also be made in accordance with the embodiment described above. The ring section 11 has a length of around 0.1 cm to 3 cm. The ring section 11 may be formed as described in the previous embodiment.

Arranged along the periphery of the annular section 11 in the longitudinal proximal direction are thin rod-like struts 13. Around 4 to 10 struts 13 are provided, spaced roughly evenly apart along the periphery of the ring section 11.

The struts 13 form a cone which tapers from the distal to the proximal end, where it has a radiopaque end ring 9 which locates all the struts 13 and connects with the filter wire 10.

Provided on the inside of the struts 13 as filter element 8 is a plastic film which has pores with a diameter of around 100 μm to 300 μm.

The size of the filter openings is chosen so that minor impurities are cleaned from the blood. Since a large filter surface is available for purifying the blood, and the purification is effected over the whole filter surface, the flow rate of the blood is affected to only a limited extent and only minimal backpressure builds up, even if the size of the filter openings is substantially constant. However, a variant with a distal filter section 6 and a proximal section 7 is preferred, wherein the proximal section has larger openings than the distal filter section 6. The distal filter section 6 extends over half to two-thirds the length of the filter 2.

The filter 2 or the filter element 8 fitted in it may be divided from distal to proximal into several sections, for example three sections. The filter element 8 of the three sections then has filter openings of increasing size from the distal to the proximal end. The filter openings of the first section have a diameter of around 100 μm to 200 μm, those of the second section a diameter of around 150 μm to 300 μm, and those of the third section a diameter of around 200 μm to 400 μm.

It is also possible to provide for a filter element 8 in the proximal section 7 to be dispensed with, so as not to impede the flow of the blood.

The filter element 8 may be made of one of the materials described above, such as e.g. PU, PTFE, PP, PE.

The different sizes of filter openings ensure the blood flowing in the arteries which supply the brain and the arms is finely filtered, and the main flow of blood in the proximal section of the filter 2 is only minimally affected by the larger filter openings of the filter element 8 or the openings of the lattice structure in this area.

Provision may also be made for the filter 2, instead of the struts, to be made of a memory material, on the inside of which a plastic film is provided as filter element 8. Moreover the lattice structure of memory material may form the filter element 8, so that the plastic film may be dispensed with.

If no plastic film is provided, then the filter 2 may be given an anti-thrombogenic coating.

The elongated, tapering filter 2 has great stiffness in the axial direction, to prevent excessive kinking of the filter in the aortic arch. The stiffness may be provided e.g. through thicker struts or by thicker struts in the lattice structure or by the arrangement of the struts of the lattice structure.

The filter 2 may also be made without the annular section 11, for example by using a lattice structure as filter 2, wherein the distal end section of the lattice structure is so designed or has the necessary stiffness that it fits tightly against the vessel wall.

In a fifth embodiment of the present invention, a filter 2 is roughly barbell-shaped (FIG. 6). At the distal and proximal ends of the filter 2, in each case a ring section 11, 12 is provided. The ring sections 11, 12 have a lattice structure and a length of around 0.5 cm to 5 cm. The lattice structure of the distal and proximal ring sections 11, 12 have the necessary stiffness to fit tightly against the wall of the blood vessel.

Provided in the area between the two ring sections 11, 12 is a tubular filter section 14. This is approximately 3 cm to 12 cm long and has owing to the design of the lattice structure only limited stiffness in the radial direction and high stiffness in the axial direction. This tubular filter section 14 has a smaller diameter than the two ring sections 11, 12.

Provided at the proximal end of the ring section 12 of the filter 2 is a tapering proximal section 7, fitted to the end of which is a radiopaque end ring 9.

Provided on the inside of the lattice structure of the tubular filter section 14 is a plastic film, which has filter openings with a diameter of around 110 μm 300 μm.

On the inside of the lattice structure of the proximal section 7 is a plastic film with filter openings larger than the filter openings of the tubular filter section and having a diameter of e.g. around 200 μm to 400 μm.

In a further embodiment of the present invention, the form of the filter is similar to that of the fifth embodiment, with a barbell-shaped filter 2. In this case, the lattice structure of the filter 2 forms the filter element 8. The size of the filter openings of the lattice structure corresponds to that of the fifth embodiment.

In all the embodiments, markers of radiopaque material may be provided at the distal end of the filter.

All of the embodiments described above may also be provided with filter openings which have a roughly constant size, with a diameter in the range of around 150 μm to 250 μm and preferably 150 μm to 200 μm over the whole length of the filter—i.e. in the distal filter section 6 as well as in the proximal section 7. This opening size is sufficiently small to filter particles from the blood flowing to the brain, while also still large enough to keep backpressure to a minimum. This applies in particular to the variant of the filter which tapers gradually from distal to proximal, since here a very good filter surface is provided. An opening size which increases towards the proximal end of the filter is however preferred.

In each of the embodiments described above, a filter wire is provided to push the filter out of the catheter. Instead of the one filter wire it is also possible to provide several filter wires or a tube-like inner catheter which is permanently connected to the filter and movable relative to a tube-like outer catheter.

In addition, the filter catheter is preferably so designed that, in the extended state of the filter, the latter emerges at the catheter lumen, i.e. the catheter tube does not extend into the filter.

In each of the above embodiments, the filter is self-expanding. Within the scope of the invention it is also possible to assist the expansion using a balloon, or to carry it out solely by the use of a balloon. Here however the options for the form of the filter are severely limited, for which reason a self-expanding filter is preferred.

The method of filtering blood is explained below. All the embodiments described above may be used for this purpose.

As shown in FIG. 1, the catheter is inserted via the ascending aorta into the aortic arch, through the femoral artery or the radial artery or the subclavian artery. The catheter is therefore inserted against the direction of blood flow, and is located in the aortic arch in the area of the bifurcation of the arteries to supply the brain and the arms.

When the filter catheter 1 is correctly positioned, the filter 2 is pushed out of the catheter lumen 5 of the catheter tube 3 through movement of the filter wire 10 from proximal to distal.

The positioning of the filter 2 may be monitored through detection of the marker at the distal end of the filter 2 or of the radiopaque end ring 9.

When a filter catheter 1 with a tubular filter 2 is used, the filter 2 expands radially in such a way that at least its distal end section is in contact with the vessel wall and preferably the entire distal filter section 6 makes contact with the vessel wall of the ascending aorta and in particular in the area of the arteries which supply the brain and the arms.

When a filter catheter 1 with a funnel-shaped filter 2 is used, the filter 2 expands radially in such a way that its distal end section is in contact with the vessel wall of the aortic arch in the area before the bifurcations of the arteries supplying the brain and the arms and fits up tightly against the vessel wall.

When a filter catheter 1 with a barbell-shaped filter 2 is used, the filter 2 expands radially in such a way that at least its distal end section or the distal ring section 11 is in contact with the vessel wall and preferably the distal and the proximal ring sections 11, 12 are correspondingly in contact with the vessel wall of the aortic arch in the area before and after the bifurcations of the arteries supplying the brain and the arms. To increase the contact pressure on the vessel wall, the distal and proximal ring sections 11, 12 of the filter 2 have high stiffness in the radial direction.

In this way it is possible to clean the blood flowing through the filter 2 from distal to proximal of embolic material, plaque and debris. In particular it is at the same time provided for the blood flowing in the arteries supplying the brain and the arms to be cleaned of especially small particles, to minimise the risk of a stroke.

Due to the coarser design of the filter 2 in its proximal section as already described, or the complete omission of a filter element in the proximal section of the filter 2, the main blood flow is affected only slightly or not at all, so that only minimal backpressure occurs. The blood is able to flow on unimpeded or with only minimal backpressure.

The filter 2 is pulled back into the catheter lumen 5 of the catheter tube 3 by movement of the filter wire 10 from distal to proximal. On pulling into the catheter lumen, the filter may remain undamaged. However, this is not necessarily the case. When the filter 2 is pulled in, the embolic material, plaque and debris filtered from the blood remain completely in the filter 2 and are pulled back with it into the catheter tube 3.

The device may of course also be inserted through any known means of access and not solely via the femoral aorta.

In the case of the device according to the invention it is advantageous that, due to the provision of a filter 2 at the distal end of the hollow catheter tube 3, wherein the filter 2 comprises a self-unfolding support structure made of a memory material, easy and rapid fitting of the filter 2 is possible.

The catheter lumen 5 of the catheter tube 3 has preferably a constant inside diameter throughout its length. It may be expedient to make the inside diameter of the catheter tube 3 in the distal end section somewhat larger than in the rest of the catheter tube 3, so that this section has sufficient space to accommodate the filter 2. The remaining area of the filter catheter 1 may not be of any desired thickness, since it must have a certain flexibility for insertion into the curved aorta. With a diameter of much more than 21 french (7 mm), this flexibility is not always guaranteed with catheter materials in common use.

All the embodiments described above may be designed so that the filter catheter has a catheter lumen through which e.g. a balloon catheter 16 may be guided. The catheter lumen of the catheter tube has a diameter of around 4 mm to 7 mm, so that the balloon catheter may be guided through it. Such a large catheter lumen allows the insertion of a balloon catheter, which may be used to remove a heart valve stenosis.

Using the balloon catheter, a dilatation balloon is first of all placed in the area of the heart valve. The dilatation balloon is guided through the heart valve by means of a guide wire. The balloon is then inflated, causing the natural heart valve to stretch open.

When the natural heart valve is stretched open by the balloon catheter, plaque and debris are released. The blood which passes during stretching open of the heart valve is filtered through the filter provided on the catheter tube located downstream of the heart valve.

The heart valve may be so strongly expanded by the balloon catheter that an artificial heart valve can be inserted. This artificial heart valve may be fed in, by means of another catheter, through the lumen of the filter catheter and placed in position. An artificial aortic valve is known e.g. from EP 1 335 683 B1. Other implantable heart valve prostheses, together with catheters for the implantation of such a heart valve prosthesis, are disclosed by EP 592 410 B1, US 2004/0210304 A1, U.S. Pat. No. 7,018,406 B2, WO2006/127765 A1, US 2003/0036795 A1, U.S. Pat. No. 5,411,552, U.S. Pat. No. 6,168,614 B1, U.S. Pat. No. 6,582,462 B1 and WO91/17720. Reference is hereby made to these documents in full.

The blood may also flow out through the catheter lumen of the catheter tube and be fed through a heart-lung machine or other external filtering device.

The catheter tube has at the end for operating the filter, the proximal end, a one-way valve (hemostasis valve) through which the balloon catheter for placing the balloon may be guided. On removal of the balloon catheter, the valve closes automatically, so that loss of blood is prevented.

Provision may also be made to suck off the material collected inside the filter, such as e.g. plaque and debris or embolism-causing material, by applying a vacuum to the proximal end of the catheter tube via the catheter lumen.

In a further advantageous embodiment, a femoral bypass is inserted, with a heart-lung machine which filters the blood being provided at the bypass. Such a device according to the invention may also be of benefit if so much plaque and debris occurs that external filtering is necessary.

In inserting a femoral bypass a catheter tube is used which has a Y-junction at its proximal end. One end of the Y-junction is provided with the one-way valve and the other has a cap which may be dissolved for connection of the blood-filtering heart-lung machine. In contrast to the prior art, the filter according to the invention is located downstream of a heart valve to be stretched open, with the elongated filter—in contrast to the prior art—extending over the aortic arch and in particular over the area in which the arteries supplying the brain and the arms are located.

The invention may be summarised briefly as follows:

The device for the filtering of blood comprises a filter catheter with a filter at its distal end for filtering the blood branching downstream behind the heart valve into the arteries to supply the brain and the arms. The filter is tubular or is designed to taper from the distal to the proximal end. The filter is so designed that, in the area downstream of the arteries which supply the brain and the arms, it fits tightly against the ascending aorta, at least at its distal end section.

### LIST OF REFERENCE NUMBERS

1 filter catheter
2 filter
3 catheter tube
4 distal end
5 catheter lumen
6 distal filter section
7 proximal section
8 filter element
9 end ring
10 filter wire
11 distal ring section
12 proximal ring section
13 struts
14 tubular section
15 area
16 balloon catheter

1. Device for the filtering of blood, comprising a filter catheter with a filter at its distal end for filtering the blood branching downstream behind the heart valve into the arteries to supply the brain and the arms, wherein the filter may be pushed out of a catheter tube in the distal direction and wherein the filter is designed to expand from the proximal to the distal in the extended state, and wherein the filter has a distal filter section and a proximal section, and the distal filter section has filter openings, and the proximal section has one or more openings which are larger than the filter openings of the distal filter section wherein a lattice structure of an end section of the distal filter section has a greater stiffness in the radial direction than in the remainder of the filter section so that the end section fits tightly against the ascending aorta.

2. Device according to claim 1, wherein the filter is longer than a section in the aortic arch in which the arteries which supply the brain and the arms branch off, subsequently described as the aortic arch section, and the distal filter section is tubular and the proximal section tapers from the distal filter section to the catheter tube, and that the filter is designed to fit tightly with its distal end section against the ascending aorta, at least in the area upstream of the arteries which supply the brain and the arms.

3. Device according to claim 1, wherein the elongated filter is designed to taper from the distal to the proximal end, and the filter is longer than a section in the aortic arch in which the arteries which supply the brain and the arms branch off, subsequently described as the aortic arch section, and that the filter is designed so as to be capable of fitting tightly with its distal end section against the ascending aorta, at least in the area upstream of these arteries.

4. Device according to any of claims 1 to 3 claim 1, wherein, characterised in that the filter (2) has a length of 5 cm to 18 cm, in particular a length of 8 cm to 16 cm or a length of 10 cm to 14 cm.

5. Device according to claim 1, wherein the lattice structure is made of memory material.

6. Device according to claim 5, wherein a filter element made of plastic film is provided on the inside or the outside of the lattice structure.

7. Device according to claim 1, wherein the filter openings in the filter section have a diameter of around 100 μm to 400 μm.

8. Device according to claim 7, wherein the filter openings along the filter increase in size from the distal to the proximal end.

9. Device according to claim 1, wherein the filter catheter has a catheter lumen which is so designed that another catheter may be guided through the catheter lumen, wherein the catheter lumen preferably has a diameter of around 4 mm to 7 mm.

10. Device according to claim 1, wherein the catheter tube is provided with a greater circumference at the distal end section in which the filter may be accommodated than in its other sections, so that the diameter of the catheter lumen is substantially constant over the entire length of the catheter tube.

11. Device according to claim 1, wherein the proximal end of the catheter tube is designed for connection to a heart-lung machine and/or an external filter.

12. Device according to claim 1, wherein a one-way valve through which another catheter may be guided is provided in the catheter lumen of the catheter tube, preferably at the proximal end of the filter catheter.

13. Device according to claim 9, wherein the other catheter is a balloon catheter, which may be guided through the catheter lumen of the catheter tube from the proximal to the distal end, wherein the balloon catheter is provided at its distal end with a balloon for the removal of a heart valve stenosis.

14. Device for the filtering of blood, comprising a filter catheter with an elongated filter at its distal end for filtering the blood branching downstream behind the heart valve into the arteries to supply the brain and the arms, wherein the filter merges at its proximal end into a catheter tube and wherein the filter may be pushed out of the catheter in the distal direction, and the filter is longer than a section in the aortic arch in which the arteries which supply the brain and the arms branch off, subsequently described as the aortic arch section, and the filter is so designed that in the area upstream of these arteries it fits tightly against the ascending aorta, at least with its distal end section, wherein the filter either tapers from the distal to the proximal end or the filter has a distal tubular filter section and a proximal section which tapers from the distal filter section to the catheter tube.

15. Device according to claim 14, wherein the filter has filter openings with a diameter of around 150 to 250 μm.

16. Method of filtering blood in the area of the arteries of the aortic arch which supply the brain and the arms, wherein a filter catheter is inserted in a vessel section downstream of the aortic arch, against the direction of blood flow, and is located in the aortic arch in the area of the arteries which supply the brain and the arms, wherein the filter has a distal filter section and a proximal section, and the distal filter section has filter openings, and the proximal section has one or more openings which are larger than the filter openings of the distal filter section and has at its distal end an expandable filter which may be pushed in and out and which is radially unfolded until at least its distal end is in contact with the vessel wall in the direction of flow before the bifurcation points of the arteries which supply the brain and the arms.

17. Method according to claim 16, wherein
after the trapping of debris and plaque in the filter, the filter is drawn into the filter catheter, and the filter catheter is then removed.

18. Method according to claim 16 wherein
a balloon catheter is guided through a catheter lumen of the filter catheter.

19. Device according to claim 1, wherein the filter openings along the filter increase in size from the distal to the proximal end.

20. Device according to claim 8, wherein the filter catheter has a catheter lumen which is so designed that another catheter may be guided through the catheter lumen, wherein the catheter lumen preferably has a diameter of around 4 mm to 7 mm.

21. Device according to claim 20, wherein the catheter tube is provided with a greater circumference at the distal end section in which the filter may be accommodated than in its other sections, so that the diameter of the catheter lumen is substantially constant over the entire length of the catheter tube.

22. Device according to claim 21, wherein the proximal end of the catheter tube is designed for connection to a heart-lung machine and/or an external filter.

23. Device according to claim 22, wherein a one-way valve through which another catheter may be guided is provided in the catheter lumen of the catheter tube, preferably at the proximal end of the filter catheter.

24. Device according to claim 23, wherein the other catheter is a balloon catheter, which may be guided through the catheter lumen of the catheter tube from the proximal to the distal end, wherein the balloon catheter is provided at its distal end with a balloon for the removal of a heart valve stenosis.

25. Device according to claim 14, wherein the filter has a distal filter section and a proximal section, and the distal filter section has filter openings, and the proximal section has one or more openings which are larger than the filter openings of the distal filter section.

26. Device according to claim 1, wherein the filter has a length of 8 cm to 16 cm

27. Device according to claim 1, wherein the filter has a length of 10 cm to 14 cm.
